(19) United States
(12) Patent Application Publication
(10) Pub. No.: US 2012/0010497 A1
(43) Pub. Date: Jan. 12, 2012

(54) Single-Sided Magnetic Resonance Imaging System Suitable for Performing Magnetic Resonance Elastography

(76) Inventors: Richard L. Ehman, Rochester, MN (US); Daniel V. Litwiller, Rochester, MN (US)

(21) Appl. No.: 13/256,160

(22) PCT Filed: Apr. 1, 2010
(86) PCT No.: PCT/US10/29556

§ 371 Date: Sep. 12, 2011

Related U.S. Application Data
(60) Provisional application No. 61/166,085, filed on Apr. 2, 2009.

(51) Int. Cl. A61B 5/055

(52) U.S. Cl. 600/410

(57) ABSTRACT

A unilateral magnetic resonance imaging (“MRI”) device (100), capable of performing magnetic resonance elastography (“MRE”) is disclosed. The unilateral MRI device includes a magnet assembly (110) that produces a static, polarizing magnetic field extending longitudinally outward from a pole face of the magnet, substantially homogeneous in a transverse plane in the near-field, and varying quasi-linearly along the longitudinal direction away from the pole face. An imaging assembly is fastened over the pole face of the magnet assembly and includes a radiofrequency (“RF”) coil (202) and a magnetic field gradient (206, 208, 210) coil that produces a magnetic field gradient in the near-field along a gradient axis. The unilateral MRI device may also include a motion source (212) to impart a vibratory motion to a subject for performing an MRE process.

### CROSS-REFERENCE TO RELATED APPLICATIONS

This application claims the benefit of U.S. Provisional Patent Application Ser. No. 61/166,085, filed on Apr. 2, 2009, and entitled “Single-Sided Magnetic Resonance Imaging Device for Magnetic Resonance Elastography.”

### BACKGROUND OF THE INVENTION

The field of the invention is magnetic resonance imaging (“MRI”) systems and methods. More particularly, the invention relates to single-sided MRI devices and magnetic resonance elastography (“MRE”).

Magnetic resonance imaging (“MRI”) uses the nuclear magnetic resonance (“NMR”) phenomenon to produce images. When a substance such as human tissue is subjected to a uniform magnetic field (polarizing field B₀), the individual magnetic moments of the nuclei in the tissue attempt to align with this polarizing field, but precess about it in random order at their characteristic Larmor frequency. If the substance, or tissue, is subjected to a magnetic field (excitation field B₁) that is in the x-y plane and that is near the Larmor frequency, the net aligned moment, M_{z}, may be rotated, or “tipped,” into the x-y plane to produce a net transverse magnetic moment M_{xy}. A signal is emitted by the excited nuclei or “spins,” after the excitation signal B₁ is terminated, and this signal may be received and processed to form an image.

When utilizing these “MR” signals to produce images, magnetic field gradients (Gₓ, G_{y}, and G_{z}) are employed. Typically, the region to be imaged is scanned by a sequence of measurement cycles in which these gradients vary according to the particular localization method being used. The resulting set of received MR signals are digitized and processed to reconstruct the image using one of many well known reconstruction techniques.

The measurement cycle used to acquire each MR signal is performed under the direction of a pulse sequence produced by a pulse sequencer. Clinically available MRI systems store a library of such pulse sequences that can be prescribed to meet the needs of many different clinical applications. Research MRI systems include a library of clinically-proven pulse sequences and they also enable the development of new pulse sequences.

The MR signals acquired with an MRI system are signal samples of the subject of the examination in Fourier space, or what is often referred to in the art as “k-space.” Each MR measurement cycle, or pulse sequence, typically samples a portion of k-space along a sampling trajectory characteristic of that pulse sequence. Most pulse sequences sample k-space in a raster scan-like pattern sometimes referred to as a “spin-warp,” a “Fourier,” a “rectilinear,” or a “Cartesian” scan. The spin-warp scan technique employs a variable amplitude phase encoding magnetic field gradient pulse prior to the acquisition of MR spin-echo signals to phase encode spatial information in the direction of this gradient. In a two-dimensional implementation (“2DFT”), for example, spatial information is encoded in one direction by applying a phase encoding gradient, G_{y}, along that direction, and then a spin-echo signal is acquired in the presence of a readout magnetic field gradient, Gₓ, in a direction orthogonal to the phase encoding direction. The readout gradient present during the spin-echo acquisition encodes spatial information in the orthogonal direction. In a typical 2DFT pulse sequence, the magnitude of the phase encoding gradient pulse, G_{y}, is incremented, ΔG_{y}, in the sequence of measurement cycles, or “views” that are acquired during the scan to produce a set of k-space MR data from which an entire image can be reconstructed.

The design of any MRI scanner typically begins with the magnet since, more than any other component, it defines and determines the imaging capabilities of the system. Despite the historic trend in clinical MR imaging toward higher field strengths and ever-larger magnets, for increased signal-to-noise ratio (“SNR”) and related improvements in resolution, field-of-view (“FOV”), and imaging time, there is also a recent, growing trend in the design of small, economical MRI systems for simple, specific applications that do not require such extreme performance. The utility of a conventional superconducting MRI system is limited, in some respects, by its reliance on a large, expensive magnet, immobile installation, fixed detector plane orientation, and finite bore size. In addition, there are other useful applications of MRI with smaller FOV requirements. For example, in applications related to imaging skin and tendons, performing bench-top pathology, or evaluating engineered tissue constructs, smaller magnets and imaging FOV may be adequate and more cost effective than the high-performance magnets typical of modern clinical MRI.

Recently, the development of MRI systems employing small, single-sided magnets has emerged, garnering attention for their relative low cost and potential for portability and handheld designs. By definition, a single-sided magnet is one in which a field suitable for imaging is produced externally to the magnet. In this arrangement, the magnet and other imaging hardware is separated from the imaging FOV by an imaginary plane, allowing the investigation of arbitrarily large surfaces using a FOV that is relatively small by conventional standards.

Currently designed magnets for single-sided MRI fall into one of several categories with respect to the various approaches employed to control homogeneity of the magnetic field. These include horseshoe-type designs that produce transverse polarizing fields, such as the one described by B. Blumich, et al., in “The NMR-Mouse: Construction, Excitation, and Applications,” Magn. Reson. Imaging, 1998; 16(5-6):479-484; simple rectilinear or cylindrical bar magnets that produce longitudinal fields, such as those described by B. Manz, et al., in “A Mobile One-Sided NMR Sensor with a Homogeneous Magnetic Field: The NMR-MOLE,” J. Magn. Reson., 2006; 183(1):25-31; and other special magnet designs. Exemplary special magnet designs include Halbach magnets, such as those described by W. Chang, et al., in “Single-Sided Mobile NMR with a Halbach Magnet,” Magn. Reson. Imaging, 2006; 24(8):1095-1102; those that incorporate field-shaping or shimming elements, such as those described by A. E. Marble, et al., in “A Constant Gradient Unilateral Magnet for Near-Surface MRI Profiling,” J. Magn. Reson., 2006; 183(2):228-34; or complex arrangements of magnets, such as those described by J. L. Paulson, et al., in Volume-Selective Magnetic Resonance Imaging Using an Adjustable, Single-Sided, Portable Sensor,” Proc. Natl. Acad. Sci. USA, 2008; 105(52):20601-20604. Static gradient strengths produced by these single-sided MRI magnets vary between of 1 and 20 Tesla-per-meter (“T/m”), with the majority of the designs falling in the 10-20 T/m range.

Current single-sided MRI devices have a list of shortcomings including low field strength and technical challenges related to radiofrequency excitation and spatial encoding. Despite this, current single-sided MRI devices that employ polarizing fields with controlled inhomogeneity are still able to produce useful, cost-effective imaging performance for their intended application.

In addition to magnet design, any MRI system must include an RF coil that produces a field with transverse components and the gradient coils that produce fields with longitudinal components that vary linearly as a function of position. In both cases, both the RF and the gradient coils are designed to produce uniform fields as efficiently as possible, thereby maximizing signal-to-noise ratio and gradient switching speeds, and minimizing power consumption. Although RF coil design receives a great deal of attention in the mainstream MRI literature, RF coil design considerations for single-sided NMR systems have been largely overshadowed by the attention given to single-sided magnet design, even though RF coil efficiency is critical at the low fields typical of single-sided systems, where intrinsic SNR is determined primarily by copper losses in the RF coil. Coil efficiency is also an important consideration for pulsed, FT-based, single-sided MRI systems because of the high peak B₁ values needed to excite usable slice bandwidths in the presence of static gradients several orders of magnitude stronger than those encountered in clinical systems.

In conventional MRI performed in a cylindrical bore magnet, RF coils are positioned with the coil normal perpendicular to B₀, which simplifies coil design and maximizes theoretical SNR, while the gradient coils are allowed to take on a volumetric shape in order to optimize the uniformity of the gradient field. However, for single-sided imaging systems that use longitudinally-polarized magnets, RF coil design can become increasingly complicated because the imaging coils are positioned in the transverse detector plane, with the coil normally positioned parallel to B₀ field.

Fortunately, for planar coil design in longitudinal single-sided imaging systems, an open-Helmholtz coil design, which includes a “figure-eight” arrangement of wire tracings, produces a field that is suitable for both the RF coil and the x and y-gradients, with a strong transverse component and a longitudinal component that vanishes at the coil center. For volumetric imaging, the z-gradient can be created by a Maxwell pair, which includes two opposing loops of wire carrying current in opposite directions. In the planar case, however, the z-gradient can be as simple as a single circular loop of wire.

The design of planar RF and gradient coils is difficult, and poses a significant challenge for single-sided MRI devices for a variety of reasons ranging from coil geometry and efficiency to coil size and sensitivity. For example, the design of planar gradient coils is a challenge for reasons primarily related to the difficulty of generating gradient fields that are linear and maximally uniform with a planar coil design.

It would therefore be advantageous to provide a small, economical MRI device capable of performing a wide variety of clinical studies on arbitrarily large surfaces, such as the skin, and for other analogous biomedical applications including the performance of bench-top pathology, the evaluation of engineered tissue, and non-destructive testing of materials.

### SUMMARY OF THE INVENTION

The present invention overcomes the aforementioned drawbacks by providing a device including a low static magnetic field gradient strength that balances between the competing needs for high through-plane resolution, short readout times, minimal chemical shift artifact, and appropriately sized fields-of-view, while maintaining relative field homogeneity in a plane transverse to the magnetic field direction.

Furthermore, the present invention provides a unilateral MRI system, or device, capable of performing magnetic resonance elastography (“MRE”). The unilateral MRI device includes a magnet assembly that produces a static, polarizing magnetic field, B₀, that extends longitudinally outward from a pole face of the magnet. In the near-field, B₀, is substantially homogenous in the transverse plane, and varies quasi-linearly along the longitudinal direction away from the pole face. An imaging assembly is fastened over the pole face of the magnet assembly includes an RF coil and at least one magnetic field gradient coil that produces a magnetic field gradient in the near-field along a gradient axis. The unilateral MRI device also includes a motion source coupled to the imaging assembly that imparts a vibratory motion to a subject such that MRE can be performed. To this end, the unilateral MRI device also includes system for driving the magnetic field gradient coil and the motion source at a selected frequency to encode received MR signals with respect to the imparted vibratory motion.

In accordance with one aspect of the invention, a unilateral MRI system is provided that includes a magnet assembly extending along a longitudinal axis from a first end to a second end and configured to produce a substantially static magnetic field extending outward from a pole face arranged at the second end of the magnet assembly and along a direction substantially parallel, in a near-field of the magnet assembly, to the longitudinal axis of the magnet assembly. The system also includes an imaging assembly connected to the pole face of the magnet assembly. The imaging assembly includes a radiofrequency (RF) coil configured to excite spins in a subject arranged within the near-field of the magnet assembly and receive MR signals from the subject, a magnetic field gradient coil configured to produce a magnetic field gradient in the near-field along a gradient axis substantially transverse to the longitudinal axis of the magnet assembly, and a magnetic-field shaping element configured to produce a magnetic field shaped to act as a blocking flux in the near-field of the magnetic assembly to control abrupt changes in flux density of the static magnetic field as a function of longitudinal distance from the forward pole face of the magnet assembly.

In accordance with another aspect of the invention, a unilateral MRI system is provided that includes a magnet assembly configured to produce a static magnetic field that extends outward from a pole face of the magnet assembly along a direction that is substantially parallel, in a near-field, to a longitudinal axis of the magnet. The system also includes an imaging assembly mounted over the pole face of the magnet assembly that includes a radiofrequency (RF) coil configured to excite spins in a subject arranged within the near-field of the magnet assembly and receive MR signals from the subject, a magnetic field gradient coil configured to produce a magnetic field gradient in the near-field along a gradient axis substantially transverse to the longitudinal axis of the magnet assembly, and a motion source configured to impart a vibratory motion to the subject. A controller is configured to control the magnetic field gradient coil and the motion source to operate at a selected frequency to encode the received MR signals with respect to the vibratory motion of the excited spins.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1A is a graphic illustration of an exemplary unilateral magnetic resonance imaging (“MRI”) device in accordance with the present invention;

FIG. 1B is an elevation view of the unilateral MRI device of FIG. 1A;

FIG. 2A is a cross section of the unilateral MRI device of FIGS. 1A and 1B;

FIG. 2B is an exploded view of an exemplary set of imaging coils that form a part of a configuration of the unilateral MRI device of FIGS. 1A and 1B;

FIG. 2C is an exploded view of an exemplary set of imaging coils, configured to include a magnetic resonance elastography (“MRE”) transducer element, that form a part of a configuration of the unilateral MRI device of FIGS. 1A and 1B;

FIG. 3A is a plan view of an exemplary spacer that forms a part of the unilateral MRI device of FIGS. 1A and 1B;

FIG. 3B is a cross section of the spacer of FIG. 3A;

FIG. 4A is a plan view of an exemplary magnetic field shaping element that forms a part of the unilateral MRI device of FIGS. 1A and 1B;

FIG. 4B is a cross section of the magnetic field shaping element of FIG. 4A;

FIG. 5A is a plan view of an exemplary nonmagnetic field shaping element that forms a part of the unilateral MRI device of FIGS. 1A and 1B;

FIG. 5B is a cross section of the nonmagnetic field shaping element of FIG. 5A;

FIG. 6 is a plan view of an exemplary structural plate that forms a part of the unilateral MRI system of FIGS. 1A and 1B;

FIG. 7 is a block diagram of an exemplary unilateral MRI system that employs the unilateral MRI device of FIGS. 1A and 1B;

FIG. 8 is a block diagram of an exemplary RF system that forms part of the unilateral MRI system of FIG. 7; and

FIG. 9 is a graphic representation of an exemplary MRE pulse sequence employed by the unilateral MRI device of FIGS. 1A and 1B and system of FIGS. 7 and 8.

### DETAILED DESCRIPTION OF THE INVENTION

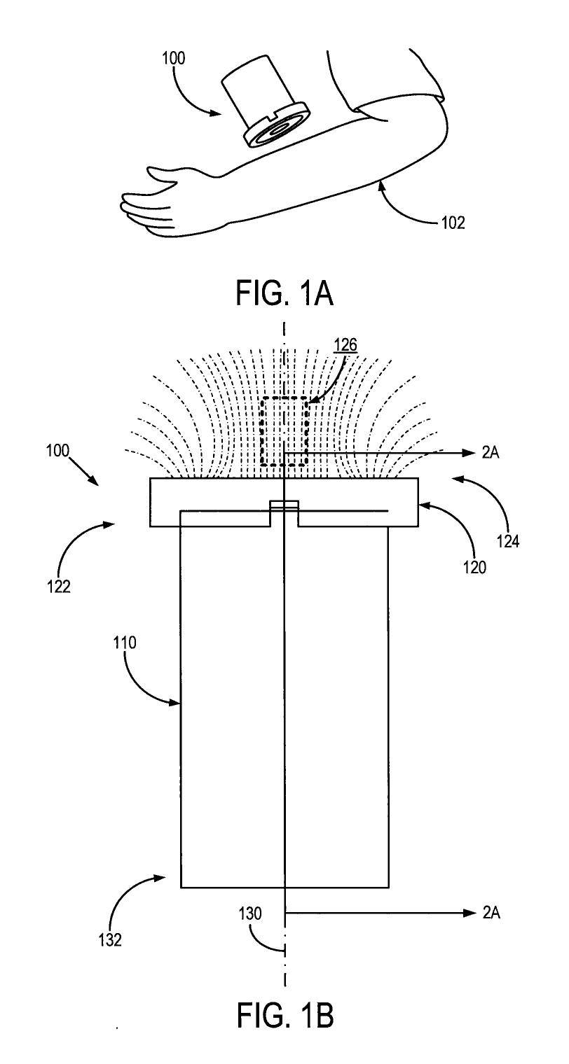

Referring to FIGS. 1A and 1B, a hand-held single-sided, or “unilateral”, magnetic resonance imaging (“MRI”) device 100 is operable to receive magnetic resonance (“MR”) image data from a subject 102. Exemplary uses include receiving MR image data from a patient's skin, a tissue sample, and an engineered tissue or other biomedical or non-biomedical materials. The unilateral MRI device 100 includes a cylindrical-shaped, bar magnet assembly 110 and an imaging assembly 120 fastened to a “forward” end 122 of the magnet assembly 110. The design of the cylindrical bar magnet 110 advantageously serves as the primary source of magnetic flux because of its simple design, ease of construction, and predictable, well-behaved magnetic field. The magnet assembly 110 and imaging assembly 120 are fastened together, as will be described in detail below, and disposed along a longitudinal axis 130 that extends from a “rearward” end 132 to the forward end of the magnet assembly 110 and passes through the center of both the magnet assembly 110 and imaging assembly 120. The magnet assembly (or electromagnet assembly) 110 may be composed of the rare earth magnetic material, neodymium-iron-boron (“NdFeB”), which advantageously provides a high magnetic remanence (proportional to magnetization). In the alternative, the magnet assembly 110 can be composed of other magnetic materials, such as samarium-cobalt (“SmCo”). In order to protect the magnet assembly 110 against oxidation and abrasion, it may be spray coated with a heat-cured phenolic resin, such as available as PR1010 from Magnet Component Engineering, of Torrance, Calif.

The overall size of the magnet assembly 110, including diameter and length, are chosen to produce a magnetic field of desired characteristics. For example, the size of the magnet assembly 110 may be chosen to produce an average static magnetic field, B₀, of 0.5 Tesla (“T”). An exemplary size of the magnet assembly 110 is a cylinder having a length of 15 centimeters (“cm”) and a diameter of 10 cm.

The cylindrical bar magnet assembly 110 is polarized in the longitudinal direction and produces at a forward pole face 124 a magnetic field 126 that has a quasi-linear field gradient directed along the longitudinal axis 130. At any distance along the longitudinal axis 130 from the forward pole face 124, this “near” magnetic field 126, or “near-field”, is relatively uniform, or homogenous, at any radial direction and distance from the longitudinal axis 130.

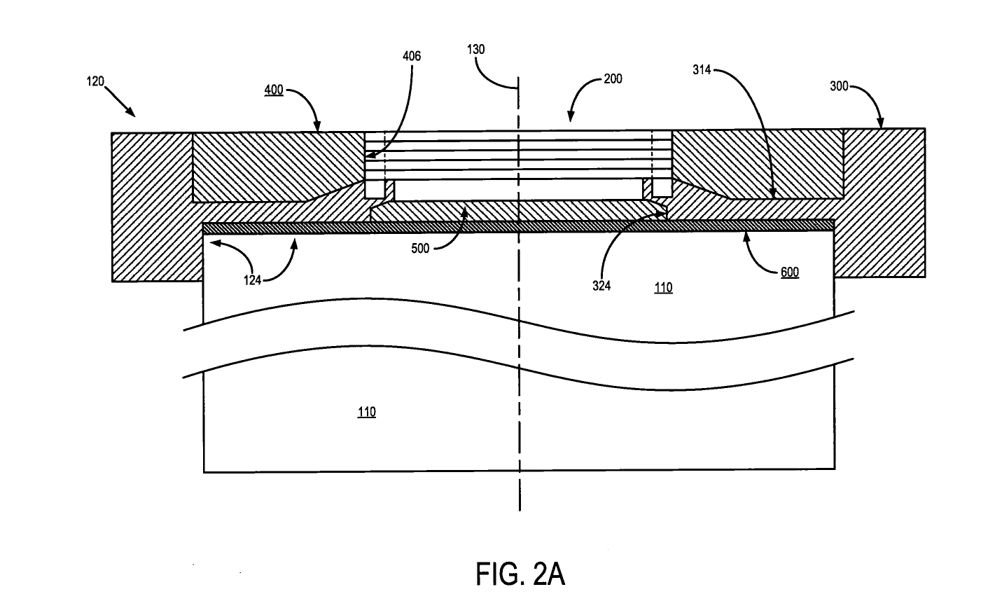

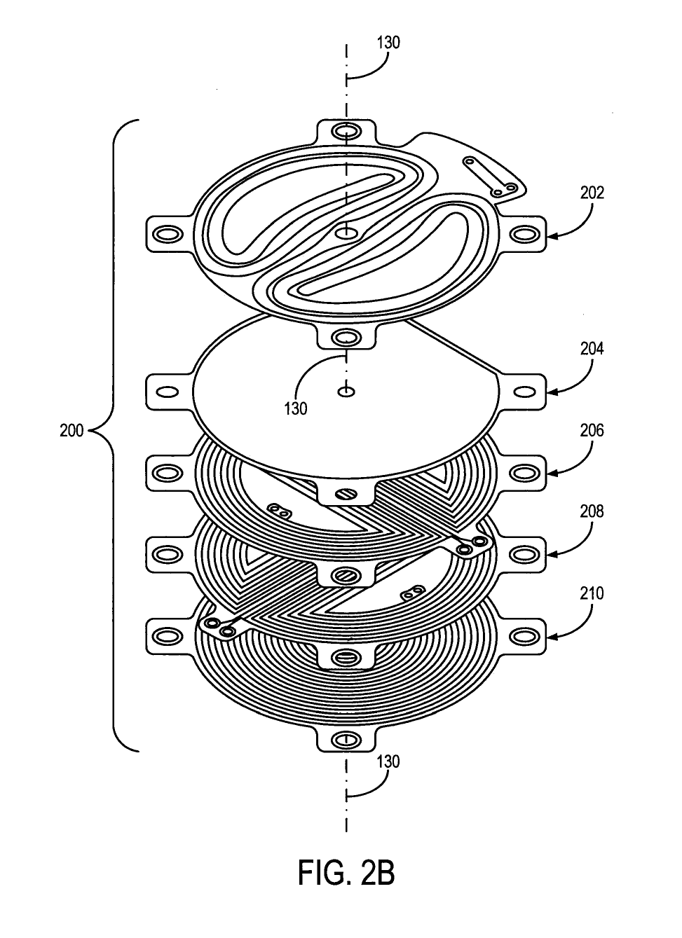

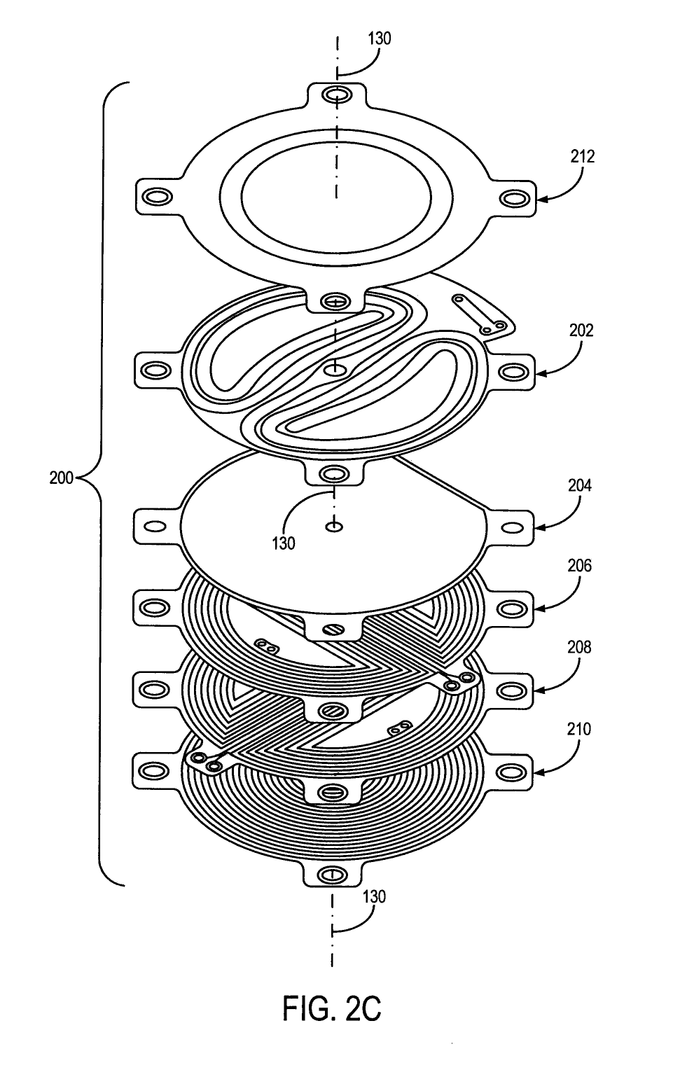

The MRI device 100 is not only suitable for traditional MR imaging procedures, but is also designed to perform a variety of useful procedures, such as clinical applications, non-destructive testing, material science research, and general research. For example, as will be described, the MRI device 100 includes imaging coils and a magnetic resonance elastography (“MRE”) vibration source, or transducer element, at the forward pole face 124 of the magnet assembly 110. To facilitate such a configuration, the imaging assembly 120 includes elements that shape its magnetic field. Referring particularly to FIG. 2A, the imaging assembly 120 includes an annular shaped spacer 300 and a disc-shaped support element 314 extends over the forward pole face 124 of the magnet assembly 110. A structural plate 600 fastens to the support element 314 with machine screws.

The support element 314 has a central opening 324 that is coaxial with the longitudinal axis 130, and which houses a disc-shaped, ferromagnetic field shaping element 500 that is retained against the surface of the structural plate 600. An annular-shaped magnetic field shaping element 400 is retained against the forward surface of support element 314 and extends radially inward from the spacer ring 300 to form a circular central bore 406 forward of the ferromagnetic field shaping element 500. The annular-shaped magnetic field shaping element 400 may be composed of the rare earth magnetic material neodymium-iron-boron (“NdFeB”). The magnetic field shaping element 400 and the magnet assembly 110 exhibit a mutual magnetic attraction that acts to hold the spacer 300, ferromagnetic field shaping elements 500, and structural plate 600 in place. The addition of these field shaping elements 400, 500 further acts to reduce the average static magnetic field, B₀, of the magnet assembly 110 from 0.5 T to 0.3 T.

Referring particularly to FIGS. 2A and 2B, a set of imaging coils 200 are mounted within the central bore 406, forward of the ferromagnetic field shaping element 500 and coaxial with the longitudinal axis 130. These imaging coils 200 include RF coils and magnetic field gradient coils, as will now be described in detail. The imaging coils are formed as layers and assembled into a stack 200, as illustrated in FIG. 2B. In order starting at its forward end, the imaging coils 200 include an RF coil 202, an RF ground plane 204, a Gₓ (“x-gradient”) coil 206, a G_{y} (“y-gradient”) coil 208, and a G_{z} (“z-gradient”) coil 210. All of the coils are disposed in a “planar” orientation with respect to the forward pole face of the magnet assembly 110 and, generally, the subject being imaged. Specifically, the Gₓ and G_{y} coils produce magnetic field gradients directed in a plane transverse to the longitudinal axis 130, and the G_{z} coil produces a magnetic field gradient directed along the longitudinal axis 130.

The design of the RF and gradient coils in a unilateral MRI device is complicated because the imaging coils are positioned in the transverse plane, with the coil normal positioned parallel to the longitudinal axis 130 and the static magnetic field, B₀. In conventional MRI performed in a cylindrical bore magnet, RF coils are positioned with the coil normal perpendicular to the direction of B₀, which simplifies coil design and maximizes theoretical signal-to-noise ratio (“SNR”), while the gradient coils are allowed to take on a volumetric shape in order to optimize the uniformity of the gradient field. To address this issue, a butterfly (or open-Helmholtz) design is employed to construct the RF, Gₓ, and G_{y} coils (202, 206, and 208). This design is chosen because, in a planar orientation, it produces an electromagnetic field with strong radial components, and longitudinal components that vanish at the coil center. Moreover, the field that is produced varies quasi-linearly with distance from the forward pole face directed along the longitudinal axis 130. The G_{z} coil 210 is constructed based on a simple planar spiral design described below.

The imaging coils 200 are fabricated on 0.020 inch two-sided printed circuit board (“PCB”) with 0.5 ounce copper cladding, immersion silver plating, and epoxy laminate insulation. The RF coil 202, an eight-turn open-Helmholtz design with a one-eight inch (3.2 millimeter) trace width, is mounted 3 millimeter (“mm”) above a circular RF ground plane 204, and tuned to 11.8 MHz and matched to 50 ohms. The thickness of the ground plane 204 is 150 micrometers (“μm”). This RF coil design allows for the slice selective excitation of spins with a slice thickness upwards of 10 mm. The Gₓ and G_{y} coils (206 and 208) are identical open-Helmholtz designs, constructed with 54 gradient windings (on-center) with a 0.040 inch trace width. The gradient coils, 206 and 208, are aligned such that their gradient fields are rotated 90 degrees with respect to each other. The G_{z} coil 210 is a simple two-sided spiral with 70 total gradient windings and a 0.040 inch trace width. Epoxy may be used to bond the gradient coils together for increased mechanical strength (for example, to resist torquing) and positioned 2 mm below the RF ground plane 204. The imaging coils 200 are assembled into a stack, positioned inside the bore 406 of the annular field shaping element 400 with the RF coil 202 flush with the forward surface 412 of the annular field shaping element 400, and then fastened to the spacer 300 with four 2-56 stainless steel machine screws. Coil cabling is passed through gaps beneath the annular field shaping element 400, as will be described below.

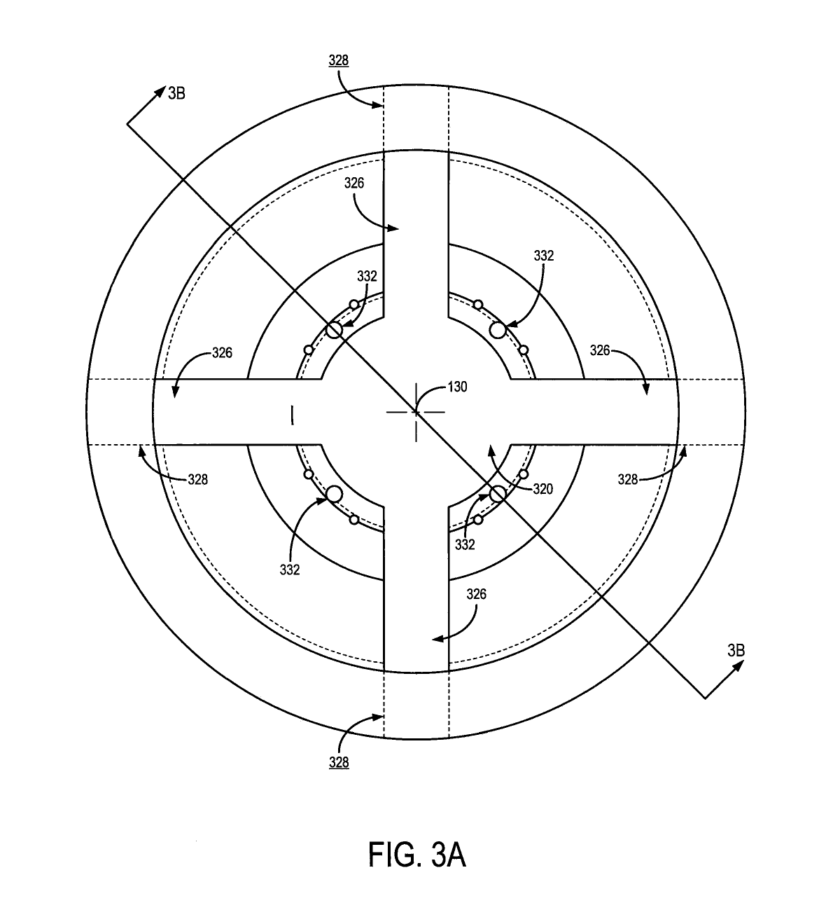

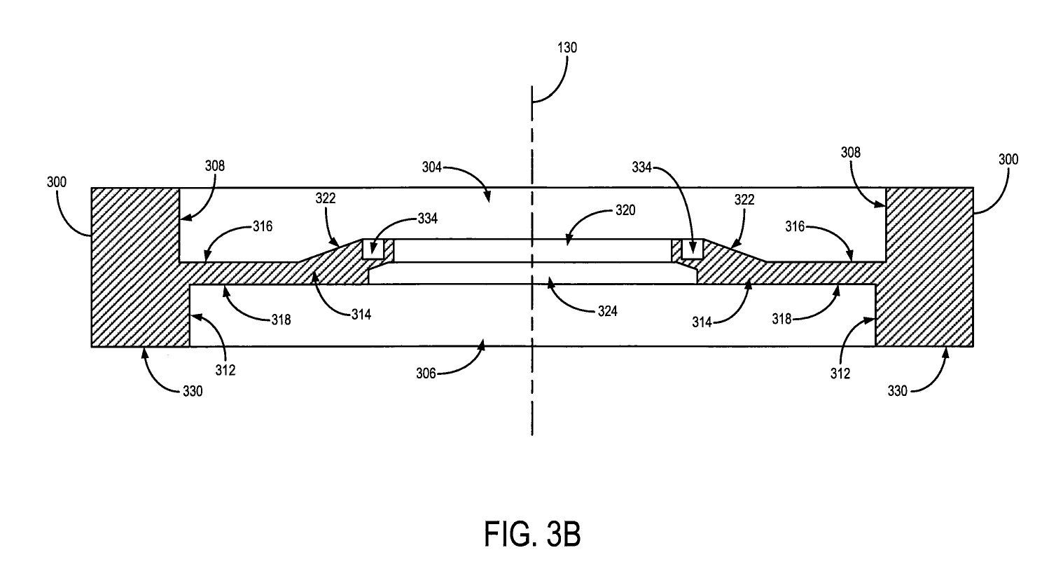

The construction of the above-described elements will now be described in more detail. Referring now particularly to FIGS. 3A and 3B, the support element 314 and annular spacer 300 are machined out of a non-magnetic material, such as the acetal resin, available under the tradename, Delrin®, which is a registered trademark of DuPont of Wilmington, Del. The annular spacer 300 is defined by a forward recessed region 304 and a rearward recessed region 306 formed with the support element 314. The forward recessed region 304 has a larger diameter than the rearward recessed region 306. The forward recessed region 304 extends from a first inner wall 308 of the spacer 300 towards the longitudinal axis 130 and the rearward recessed region 306 extends from a second inner wall 312 of the spacer 300 towards the longitudinal axis 130. The forward recessed region 304 and the rearward recessed region 306 are separated by the support element 314, which is integrally formed with the spacer 300.

The support element 314 has a forward surface 316 that extends from the first inner wall 308 of the spacer 300 towards the longitudinal axis 130 and a rearward surface 318 that extends from the second inner wall 312 of the spacer 300 towards the longitudinal axis 130, thereby circumscribing a central bore 320. The portion of the forward surface 316 of the support element 314 that circumscribes the central bore 320 is raised and encircled by a chamfered edge 322. The forward recessed region 304 is formed in this manner so that the annular field shaping element 400 contacts the forward surface 316 of the support element 314 and circumscribes the chamfered edge 322. A central recessed region 324 having a diameter larger than the central bore 320 extends from the rearward surface 318 of the support element 314 towards the forward surface 316 of the support element 314. The central recessed region 324 is formed so as to receive the ferromagnetic field shaping element 500 such that it is circumscribed by the support element 314.

The support element 314 is partitioned into four equal sectors by two orthogonal channels 326 (FIG. 3A) that extend from one side of the spacer 300 to the other and from a rearward surface 330 of the spacer 300 to the forward surface of the support element 314. In this manner, the channels form four pass-through regions 328 (FIG. 3A) in the spacer 300 such that cables can be passed from a pass-through 328 to the central bore 320, where they connect with the imaging coils 200. Threaded inserts 332 are placed in the forward surface 316 of the support element 314 between the chamfered edge 322 and central bore 320. One such threaded insert 332 is placed in each partitioned sector of the support element 314. The threaded inserts 332 are utilized to fasten the imaging coils 200 to the support element 314, as discussed above.

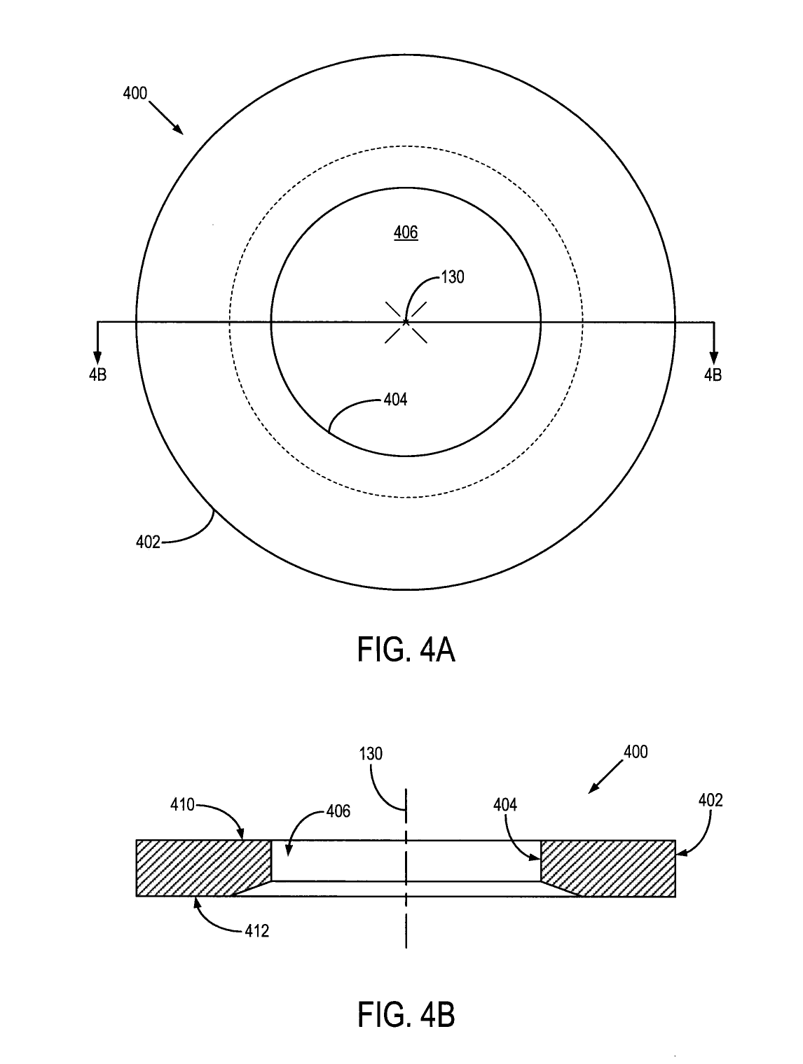

Referring now particularly to FIGS. 4A and 4B, the annular field shaping element 400 is a magnetic ring having a circular cylindrical shaped outer surface 402 and a circular cylindrical inner surface 404 that defines a central bore 406. The annular field shaping element 400 is sized such that the first inner wall 308 of the spacer 300 circumscribes the outer surface 402 of the annular field shaping element 400, as illustrated in FIG. 2A. The inner surface 404 of the annular field shaping element 400 is chamfered toward a rearward surface 410 thereof, such that the chamfered portion of the inner surface 404 circumscribes the chamfered edge 322 of the support element 314, as illustrated in FIG. 2A. The annular field shaping element 400 may be composed of the rare earth magnetic material neodymium-iron-boron (“NdFeB”), and produce a magnetic field that extends in its near-field from its forward surface 412 in a direction substantially parallel with the longitudinal axis 130. As with the magnet assembly 110, the annular field shaping element 400 can be alternatively composed of other magnetic or electromagnet materials, such as samarium-cobalt (“SmCo”). Similarly, the annular field shaping element 400 may be coated in a heat-cured phenolic resin to protect against oxidation and abrasion. As described above, the annular field shaping element 400 is retained against the forward surface 316 of the support element 314 by the mutual magnetic attraction between the annular field shaping element 400 and the magnet assembly 110. In general, the configuration of the annular field shaping element 400 and its position with respect to the magnet assembly 110 provides a “blocking” flux in the near-field 126. This arrangement prevents the flux density from falling off precipitously as a function of longitudinal distance from the forward pole face of the magnet assembly 110 in the near field 126.

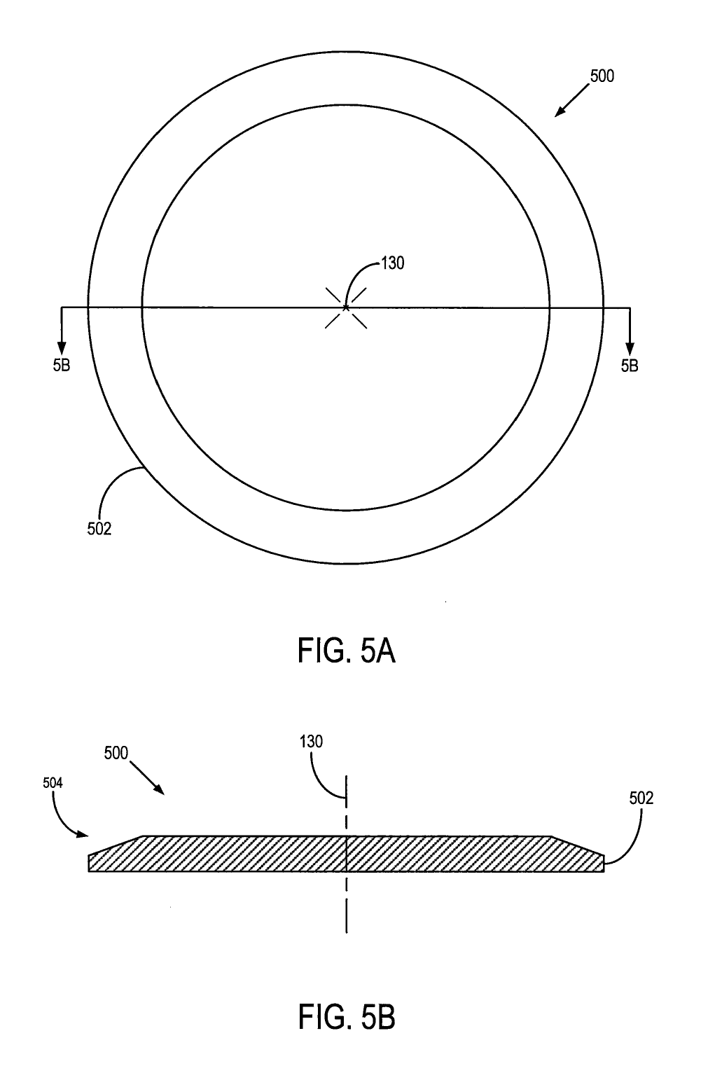

Referring particularly now to FIGS. 5A and 5B, the ferromagnetic field shaping element 500 is a disc-shaped element that may be, for example, composed of low-carbon steel and having a circular cylindrical outer surface 502 that is chamfered on a forward end 504. The ferromagnetic field shaping element 500 is also annealed to remove grain coarseness, thereby substantially mitigating local magnetic field anomalies. As with the magnet assembly 110 and the annular field shaping element 400, the ferromagnetic field shaping element 500 may be coated in a heat-cured phenolic resin to protect against oxidation and abrasion.

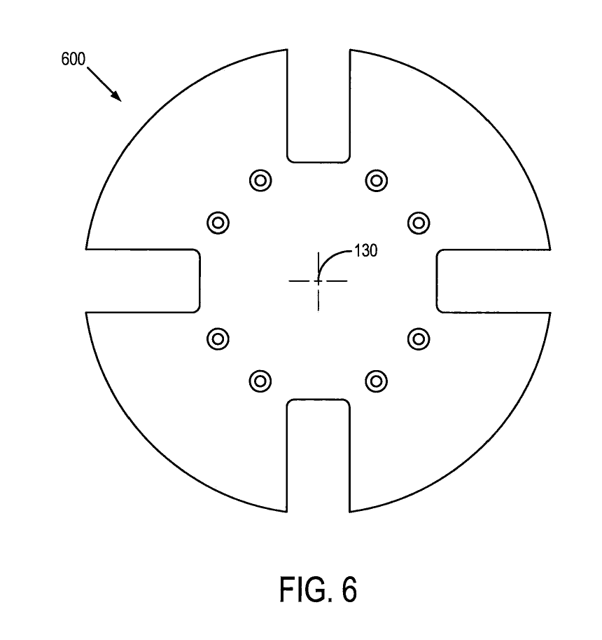

As described above with respect to FIGS. 2A and 3B, the ferromagnetic field shaping element 500 is positioned in the central recessed region 324 of the support element 314 such that the support element 314 circumscribes the ferromagnetic field shaping element 500 and the chamfered edge of the ferromagnetic field shaping element 500 engages the rearward surface 318 of the support element 314. As illustrated in FIG. 2A, the ferromagnetic field shaping element 500 is held in place between a structural plate 600, such as the one shown in FIG. 6, and the support element 314. The structural plate may be composed of flame retardant-4 (“FR-4”) printed circuit board (“PCB”) and is fastened to the support element 314 with screws through holes 334. The ferromagnetic field shaping element 500 distributes the magnetic field flux produced by the magnet assembly 110 evenly across the bottom of the near-field 126. In this manner, a substantially homogenous magnetic field is produced, in the near-field 126, in planes transverse to the longitudinal axis 130. In sum, the field shaping elements, 400 and 500, interact with the static magnetic field produced by the magnet assembly 110 such that the gradient of the magnetic field is reduced by an order of magnitude, while preserving average field strength and substantial field homogeneity in directions extending perpendicularly away from the longitudinal axis 130.

In another configuration, referring to FIG. 2C, a magnetic resonance elastography (“MRE”) transducer element 212 is also included in the unilateral MRI system 100. Exemplary MRE transducer element 212 includes an external bending element 214, a piezoelectric disc 216 disposed within the bore 406 of the annular field shaping element 400 and flush with the forward surface 412 of the annular field shaping element 400, and a flat piezoelectric extension motor (not shown). More particularly, the piezoelectric disc 216 may be positioned beneath the coils 200 and beneath an RF shield. The extension motor may be positioned off to the side of the unilateral MRI system 100 and flush with the forward surface 412 or may be arranged lengthwise in the bore 406. Also, the external bending element 214 may be include a piezoelectric element, an electromechanical element, pneumatic element, and the like. Such a configuration of the unilateral MRI system 100 enables the performance of magnetic resonance elastography (“MRE”). An exemplary configuration in which a piezoelectric disc is employed as the MRE transducer element 212, and is integrated into the stack of imaging coils 200, is shown in FIG. 2C.

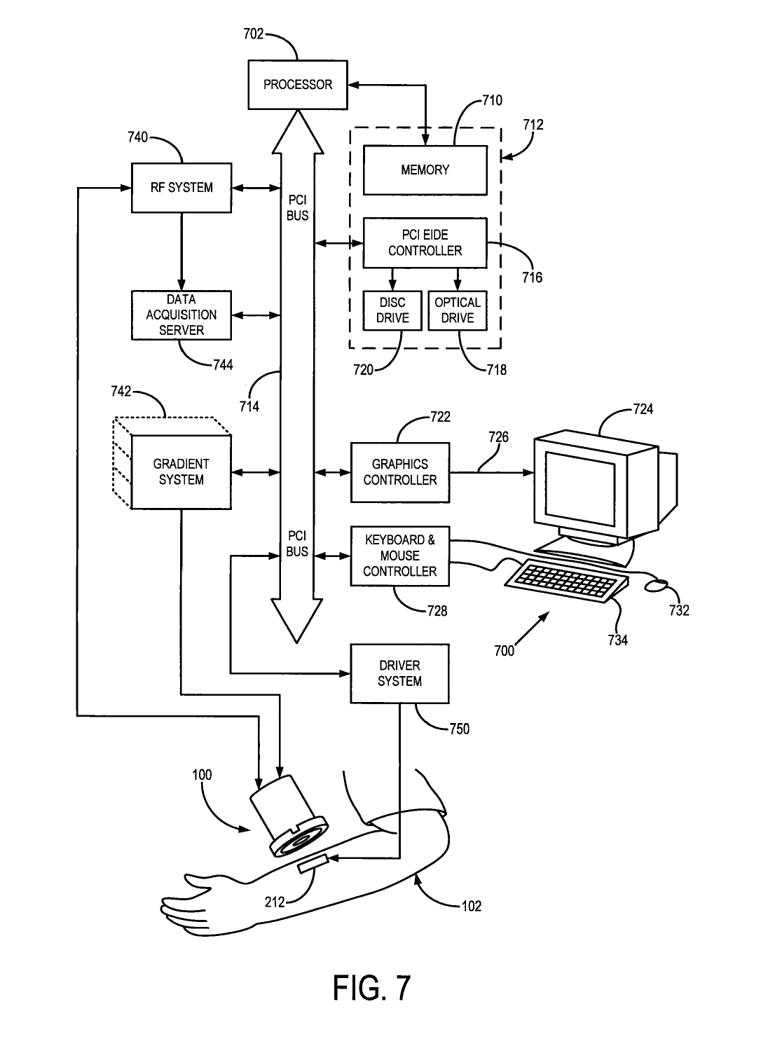

Referring particularly to FIG. 7, the preferred embodiment of the present invention employs an imaging system that includes a workstation 700, which provides an operator interface that enables scan prescriptions to be passed to the unilateral MRI device 100. The computer workstation 700 includes a processor 702 that executes program instructions stored in a memory 710, which forms part of a storage system 712. The processor 702 is a commercially available programmable machine running a commercially available operating system. It includes internal memory and I/O control to facilitate system integration and integral memory management circuitry for handling all external memory 710. The processor 702 also includes a PCI bus driver which provides a direct interface with a PCI bus 714.

The PCI bus 714 is an industry standard bus that transfers data between the processor 702 and a number of peripheral controller cards. These include a PCI EIDE controller 716 which provides a high-speed transfer of data to and from an optical drive 718 and a disc drive 720. A graphics controller 722 couples the PCI bus 714 to a monitor 724 through a standard display connection 726, and a keyboard and a mouse controller 728 receives data that is manually input through a keyboard 730 and mouse 732. The PCI bus 714 also connects to a radiofrequency system 740 and a gradient system 742.

The processor 702 acts in part as a pulse sequencer and functions in response to instructions downloaded from the workstation 700 to operate the RF system 740 and the gradient system 742. Gradient waveforms necessary to perform the prescribed scan are produced and applied to the gradient system 740 that excites gradient coils (206, 208, and 210) in the unilateral MRI device 100 to produce the magnetic field gradients Gₓ, G_{y}, and G_{z} used for position encoding MR signals. The gradient system 742 includes, for example, a set of high-power, open-frame operational-amplifiers (models MP111, MP230, Apex Precision Power, Cirrus Logic, Austin, Tex.), wired in a current-sense feedback configuration and powered with a pair of +48 VDC power supplies (Power-One FNP1500-48, Camarillo, Calif.), combined to provide ±48 VDC.

RF excitation waveforms are applied to the RF coil 202 by the RF system 740 to perform the prescribed magnetic resonance pulse sequence. Responsive MR signals detected by the RF coil 202 are received by the RF system 740, amplified, demodulated, filtered, and digitized under direction of commands produced by the processor 702. The RF system 740 includes an RF transmitter for producing a wide variety of RF pulses used in MR pulse sequences. The RF transmitter is responsive to the scan prescription and direction from the processor 702 to produce RF pulses of the desired frequency, phase, and pulse amplitude.

The RF system 740 also includes one or more RF receiver channels. Each RF receiver channel includes an RF amplifier that amplifies the MR signal received by the coil to which it is connected and a detector that detects and digitizes the I and Q quadrature components of the received MR signal. The magnitude of the received MR signal may thus be determined at any sampled point by the square root of the sum of the squares of the I and Q components:

M=\sqrt{I²+Q²} Eqn. (1);

and the phase of the received MR signal may also be determined:

$$φ = {tan}^{- 1} ( \frac{Q}{I} ) . \qquad Eqn . ( 2 )$$

The digitized MR signal samples produced by the RF system 740 are received by a data acquisition server 744. The data acquisition server 744 operates in response to instructions downloaded from the workstation 700 to receive the real-time MR data and provide buffer storage such that no data is lost by data overrun. In some scans, the data acquisition server 744 does little more than pass the acquired MR data to the processor 702. However, in scans that require information derived from acquired MR data to control the further performance of the scan, the data acquisition server 744 is programmed to produce such information and convey it to the processor 702. For example, during prescans, MR data is acquired and used to calibrate the pulse sequence performed by the pulse sequencer.

The processor 702 receives MR data from the data acquisition server 744 and processes it in accordance with instructions downloaded from the workstation 700. Such processing may include, for example: Fourier transformation of raw k-space MR data to produce one-, two-, or three-dimensional images; the application of filters to a reconstructed image; the performance of a backprojection image reconstruction of acquired MR data; the calculation of functional MR images; the calculation of motion or flow images; and the calculation of MRE wave images and elastograms.

Images reconstructed by the processor 702 are conveyed back to the storage system 712, where they are stored. Real-time images are stored in a data base memory cache 710, from which they may be output to operator display 724. Batch mode images or selected real-time images are stored in an optical drive 718 or disc drive 720.

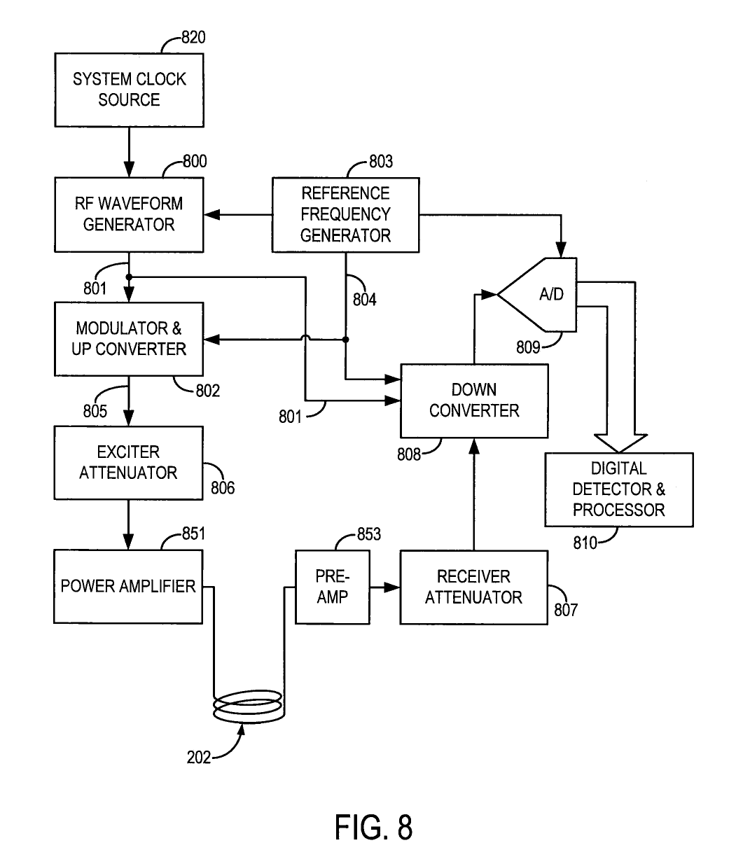

The radiofrequency (“RF”) system 740 is connected to the RF coil 202. Referring now particularly to FIG. 8, the RF system 740 includes a transmitter that produces a prescribed RF excitation field. The base, or carrier, frequency of this RF excitation field is produced under control of an RF waveform generator 800 (DA4300, Chase Scientific) that receives a set of digital signals from the pulse sequencer in the processor 702. These digital signals indicate the frequency and phase of the RF carrier signal produced at an output 801. The RF carrier is applied to a modulator and up converter 802 where its amplitude is modulated in response to a signal, R(t), also received from the pulse sequencer in the processor 702. The signal, R(t), defines the envelope of the RF excitation pulse to be produced and is produced by sequentially reading out a series of stored digital values. These stored digital values may be changed to enable any desired RF pulse envelope to be produced. To ensure synchronous operation of the RF system 740, which is necessary to maintain phase coherence, clock signals derived from a single system clock source 820 (CG400, Chase Scientific, Langley, Wash.) are provided to the RF waveform generator 800.

The magnitude of the RF excitation pulse produced at output 805 is attenuated by an exciter attenuator circuit 806 that receives a digital command from the pulse sequencer in the processor 702. The attenuated RF excitation pulses are applied to the power amplifier 851 that drives the RF coil 202.

Referring still to FIG. 8 the signal produced by the subject is picked up by the RF coil 202 and applied through a preamplifier 853 to the input of a receiver attenuator 807. The receiver attenuator 807 further amplifies the signal by an amount determined by a digital attenuation signal received from the pulse sequencer in the processor 702. The received signal is at or around the Larmor frequency, and this high frequency signal is down converted in a two step process by a down converter 808 that first mixes the MR signal with the carrier signal on line 801 and then mixes the resulting difference signal with a reference signal on line 804. The down converted MR signal is applied to the input of an analog-to-digital (A/D) converter 809 that samples and digitizes the analog signal and applies it to a digital detector and signal processor 810 that produces 16-bit in-phase (I) values and 16-bit quadrature (Q) values corresponding to the received signal. The resulting stream of digitized I and Q values of the received signal are output to the data acquisition server 744. The reference signal, as well as the sampling signal applied to the A/D converter 809, are produced by a reference frequency generator 803.

Referring again particularly to FIG. 7, the MRE transducer element 212 may be, in one configuration, supplied external to, the unilateral MRI device 100. However, as described above with respect to FIG. 2C, the MRE transducer element 212 may be integrated with the MRI device 100. In either case, the MRE transducer is driven by a driver system 750, such as a frequency generator. As will be described below, the MRE transducer element 212 produces a vibratory motion, or oscillatory stress, in the subject 150 that provides a phase contrast mechanism by which MRE is performed.

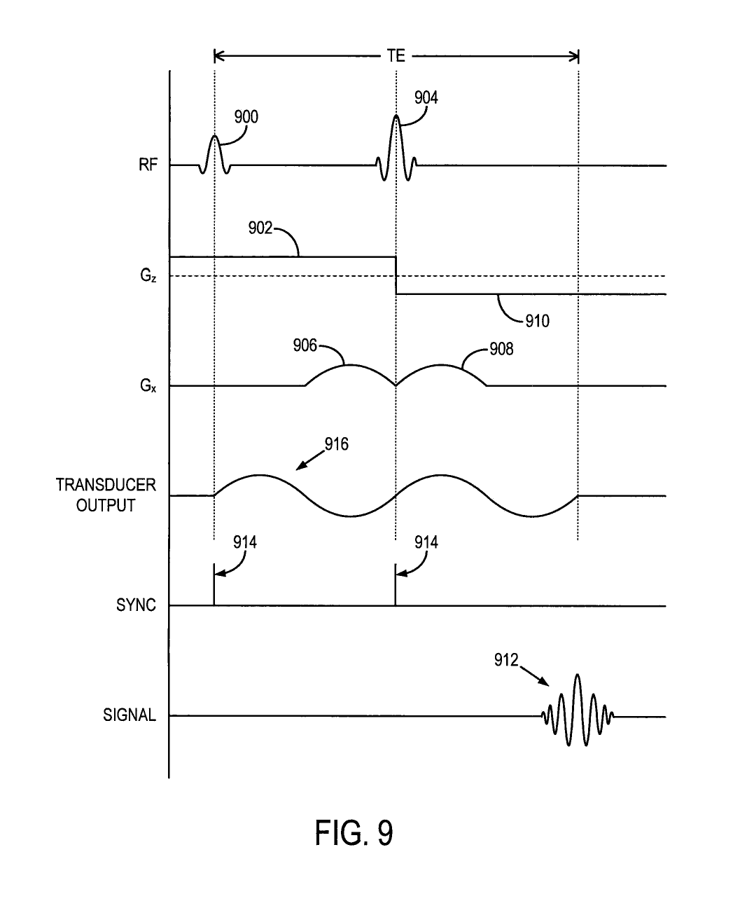

Referring particularly to FIG. 9, an exemplary pulse sequence, which may be used to acquire magnetic resonance (“MR”) data according to an embodiment of the present invention, is shown. The pulse sequence is fundamentally a 2DFT pulse sequence using a spin echo. Transverse magnetization is produced by a selective 90 degree radiofrequency (“RF”) excitation pulse 900 that is produced in the presence of a slice selective gradient, which is the effective G_{z} 902. Generally speaking, the static magnetic field produced by the magnet assembly 110 has a linear gradient along the longitudinal axis (“G_{z}-axis”), which is utilized as the slice selective gradient 902. It will be appreciated by those skilled in the art, however, that the magnetic field gradient coils (206, 208, and 210) can also be employed to modify the slice selective gradient field. Subsequently, a 180 degree refocusing RF pulse 904 is applied, which effectively reverses the direction of the static gradient G_{z}. A first motion encoding gradient lobe 906 is applied prior to the refocusing RF pulse 904, and a second motion encoding gradient lobe 908 is applied thereafter. The linear gradient of the static magnetic field is then utilized as a readout gradient 910. After a duration of time referred to as the echo time (“TE”) has passed since the application of the RF excitation pulse 900, a so-called “spin echo” is formed. A resultant MR signal 912 is detected from the formation of the spin echo. As will be appreciated by those skilled in the art, phase encoding gradients can also be applied with the magnetic field gradient coils (206, 208, and 210) so that the acquired MR signal 912 may be spatially or motion encoded.

The alternating magnetic field gradients 906 and 908 are applied after the transverse magnetization is produced and before the MR signal is acquired. The motion encoding gradients, 906 and 908, are considered “alternating” since the refocusing RF pulse 904 effectively inverts the polarity of the second motion encoding gradient 908. In the pulse sequence illustrated in FIG. 9, the alternating magnetic field gradients 906 and 908 are applied along the Gₓ-axis. As noted above, the polarity of the two gradients 906 and 908 are effectively alternated by the refocusing RF pulse 904, which results in an effective bipolar gradient waveform. The frequency of the alternating gradients 906 and 908 is set to the same frequency used to drive the magnetic resonance elastography (“MRE”) transducer element 212. At the same time, the pulse sequencer in the processor 702 produces sync pulses as shown at 914, which have the same frequency as, and have a specific phase relationship with respect to, the alternating gradient pulses 906 and 908. These sync pulses 914 are provided to the driver system 750 and used to produce the drive signals for the MRE transducer element 212. In this manner, the MRE transducer 212 is directed to apply an oscillating stress 916 to the subject. To ensure that the resulting waves have time to propagate throughout the field of view, the sync pulses 914 may be turned on well before the pulse sequence begins, as shown in FIG. 9.

The phase of the MR signal 912 is indicative of the movement of the spins. If the spins are stationary, the phase of the MR signal is not altered by the alternating gradient pulses 906 and 908, whereas spins moving along the motion encoding gradient axis (Gₓ-axis) will accumulate a phase proportional to their displacement. Spins which move in synchronism and in phase with the alternating magnetic field gradients 906 and 908 will accumulate maximum phase of one polarity, and those which move in synchronism, but 180 degrees out of phase with the alternating magnetic field gradients 906 and 908, will accumulate maximum phase of the opposite polarity. The phase of the acquired MR signal 912 is thus affected by the “synchronous” movement of spins along the Gₓ-axis.

The pulse sequence in FIG. 9 can be modified to measure synchronous spin movement along the other gradient axes (G_{y} and G_{z}). For example, the alternating magnetic field gradient pulses may be applied along the G_{y}-axis, or they may be applied along the G_{z}-axis. Indeed, they may be applied simultaneously to two or three of the gradient field directions to “read” synchronous spin movements along any desired direction. It will be appreciated by those skilled in the art that many different pulse sequences can be employed with the present invention.

The material properties of tissue are measured using MRE by applying a stress and observing the resulting strain. For example a tension, pressure, or shear is applied to a subject and the resulting elongation, compression, or rotation is observed. By measuring the resulting strain, material properties of the tissue such as Young's modulus, Poisson's ratio, shear modulus, and bulk modulus can be calculated. Moreover, by applying the stress in all three dimensions and measuring the resulting strain, the material properties of the tissue can be completely defined. Exemplary methods for producing images indicative of the material properties of a subject, or so-called “elastograms,” using MRE are described, for example, in U.S. Pat. No. 5,592,085, which is herein incorporated by reference in its entirety.

Therefore, a unilateral MRI device is provided that is configured for application to MRE. The unilateral MRI device further includes field shaping elements, the configuration of which reduce the static magnetic field gradient by an order of magnitude, while maintaining as much of the radial uniformity and average field strength as possible. Although near perfect field homogeneity is considered ideal for conventional MRI, given the high through-plane resolution required for MRI of the skin and other layered surfaces, the controlled inhomogeneity provided by this configuration is advantageous for imaging skin. This is because the produced field exhibits a constant longitudinal gradient and relative radial homogeneity, providing high through-plane resolution, high through-plane k-space velocities, and minimal chemical shift artifacts. These properties are important for high-resolution imaging of short T₂ species, such as the dermis.

Furthermore, a unilateral MRI device with a relatively low static magnetic field gradient is provided. Although the strong static gradient typical of most single-sided imaging devices provides for high through-plane resolution and minimal chemical shift artifact, the field also causes signal attenuation when significant levels of molecular diffusion are present. This typically occurs in soft, water-based materials, which further limits the duration of the available MR signal, establishing an upper limit on echo time (“TE”) and echo train lengths. The echo attenuation due to diffusion has specific implications for MRE applications since the minimum TE is typically determined by an integer multiple of the temporal period of the applied motion, limiting the frequency and number of the motion encoding gradient pairs.

The present invention has been described in terms of one or more preferred embodiments, and it should be appreciated that many equivalents, alternatives, variations, and modifications, aside from those expressly stated, are possible and within the scope of the invention.

1. A unilateral magnetic resonance imaging (MRI) system comprising:
a magnet assembly extending along a longitudinal axis from a first end to a second end and configured to produce a substantially static magnetic field extending outward from a pole face arranged at the second end of the magnet assembly and along a direction substantially parallel, in a near-field of the magnet assembly, to the longitudinal axis of the magnet assembly;
an imaging assembly connected to the pole face of the magnet assembly and including:
a radiofrequency (RF) coil configured to excite spins in a subject arranged within the near-field of the magnet assembly and receive MR signals from the subject;
a magnetic field gradient coil configured to produce a magnetic field gradient in the near-field along a gradient axis substantially transverse to the longitudinal axis of the magnet assembly; and
a magnetic-field shaping element configured to produce a magnetic field shaped to act as a blocking flux in the near-field of the magnetic assembly to control abrupt changes in flux density of the static magnetic field as a function of longitudinal distance from the forward pole face of the magnet assembly.

2. The unilateral MRI system of claim 1 further comprising another magnetic-field shaping element configured to distribute a magnetic flux produced by the magnet assembly evenly across the near-field of the magnet assembly.

3. The unilateral MRI system of claim 2 wherein the another magnetic-field shaping element is configured to produce a magnetic field that extends along the near-field of the magnet assembly in a direction substantially parallel with the longitudinal axis.

4. The unilateral MRI system of claim 2 wherein the magnetic-field shaping element and the another magnetic-field shaping element are configured to interact with the static magnetic field produced by the magnet assembly such that the magnetic field gradient is reduced by an order of magnitude, while preserving an average field strength and substantial field homogeneity in directions extending perpendicularly away from the longitudinal axis.

5. The unilateral MRI system of claim 1 wherein the magnetic-field shaping element is retained against the pole face of the magnet assembly by a mutual magnetic attraction between the magnetic-field shaping element and the magnet assembly.

5. The unilateral MRI system of claim 1 further comprising a motion source configured to impart a vibratory motion to the subject and a controller configured to control the magnetic field gradient coil and the motion source at a selected frequency to encode the received MR signals with respect to the vibratory motion of the excited spins.

6. The unilateral MRI system 5 wherein the motion source is coupled to the magnet assembly.

7. The unilateral MRI system of claim 6 wherein the motion source includes a magnetic resonance elastography (MRE) transducer including a bending element.

8. The unilateral MRI system of claim 7 wherein the bending element includes a piezoelectric disc disposed within a bore extending through magnetic-field shaping element and a substantially-flat, piezoelectric extension motor.

9. The unilateral MRI system of claim 5 wherein the motion source is coupled to the subject.

10. The unilateral MRI system of claim 1 wherein the magnetic-field shaping element includes a circular cylindrical shaped outer surface and a circular cylindrical inner surface that defines a central bore extending through the magnetic-field shaping element.

11. The unilateral MRI system of claim 10 further comprising another magnetic-field shaping element configured to distribute a magnetic flux produced by the magnet assembly evenly across the near-field of the magnet assembly, wherein the another magnetic-field shaping element forms a disc having a center substantially aligned within the central bore extending through the magnetic-field shaping element and retained against the pole face of the magnet assembly by a mutual magnetic attraction between the magnetic-field shaping element and the magnet assembly.

12. A unilateral magnetic resonance imaging (MRI) system comprising:
a magnet assembly configured to produce a static magnetic field that extends outward from a pole face of the magnet assembly along a direction that is substantially parallel, in a near-field, to a longitudinal axis of the magnet;
an imaging assembly mounted over the pole face of the magnet assembly comprising:
a radiofrequency (RF) coil configured to excite spins in a subject arranged within the near-field of the magnet assembly and receive MR signals from the subject;
a magnetic field gradient coil configured to produce a magnetic field gradient in the near-field along a gradient axis substantially transverse to the longitudinal axis of the magnet assembly;
a motion source configured to impart a vibratory motion to the subject; and
a controller configured to control the magnetic field gradient coil and the motion source to operate at a selected frequency to encode the received MR signals with respect to the vibratory motion of the excited spins.

13. The unilateral MRI system of claim 12 further comprising a magnetic-field shaping element configured to shape the static magnetic field and the magnetic field gradient to be substantially homogenous in a plane transverse to the longitudinal axis and decrease substantially linearly with distance from the pole face of the magnet assembly in the near-field.

14. The unilateral MRI system of claim 13 wherein the magnetic field shaping element includes:
a) a non-magnetic support structure; and
b) a ferromagnetic field shaping element disposed between the pole face of the magnet assembly and the support structure and in a plane substantially perpendicular to the longitudinal axis.

15. The unilateral MRI system of claim 14 further comprising an annular magnetic field shaping element mounted to the support structure and encircling the ferromagnetic field shaping element in a plane forward of the ferromagnetic field shaping element

16. The unilateral MRI system of claim 13 wherein the ferromagnetic field shaping element and annular magnetic field shaping element configured to interact with the magnet assembly to produce a static magnetic field that extends outward from a pole face along a direction that is substantially parallel, in the near-field, to the longitudinal axis.

17. The unilateral MRI system of claim 15 wherein the ferromagnetic field shaping element is retained against the pole face of the magnet assembly by a mutual magnetic attraction between the annular magnetic field shaping element and the magnet assembly.

18. The unilateral MRI system of claim 13 wherein the motion source is coupled to magnet assembly through the magnetic field shaping element, RF coil, and magnetic field gradient coil.

19. The unilateral MRI system of claim 12 wherein motion source includes a magnetic resonance elastography (MRE) transducer including a piezoelectric element.

20. The unilateral MRI system of claim 19 wherein the piezoelectric element includes a piezoelectric disc disposed within a bore extending through magnetic-field shaping element.
